(19) United States
(12) Patent Application Publication
(10) Pub. No.: US 2006/0008895 A1
(43) Pub. Date: Jan. 12, 2006

(54) Microarray chip for detection of immunoglobulin

(76) Inventors: Hsiu-Mei Chen, Taipei Hsien (TW); Jiu-Yao Wang, Taipei Hsien (TW); Li-Chuan Chen, Taipei Hsien (TW); Sen-Lin Lo, Taipei Hsien (TW); Hsiu-Wen Lee, Taipei Hsien (TW)

Correspondence Address: SUPREME PATENT SERVICES, POST OFFICE BOX 2339, SARATOGA, CA, 95070, US

(21) Appl. No.: 10/887,656

(22) Filed: Jul. 8, 2004

(51) Int. Cl. C12M 1/34

(52) U.S. Cl. 435/287.2

(57) ABSTRACT

Disclosed is a microarray chip for allergy-related immunoglobulin detection, especially for quantitative detection of total IgE and allergen-specific immunoglobulins (such as specific IgE, specific IgG, and specific IgM), which comprises a solid substrate, a reactive layer fabricated on the solid substrate, and at least one allergen or substance capable of binding to immunoglobulin of interest. Whereby, use the result of quantitative detection for allergen-specific IgE to determine hypersensitivity level. In addition, a method for allergy-related immunoglobulin detection using the microarray chip is present, which uses a secondary monoclonal antibody to minimize non-specific binding and applies an enzymatic reaction to amplify reaction signal. An efficient way is thus obtained, which not only reduces time consumption but also provides quantitative measurement.

### BACKGROUND OF THE INVENTION

1. Field of the Invention

The present invention relates to a microarray chip for immunoglobulin detection and especially for simultaneous detection of both total IgE and allergen-specific immunoglobulins in a sample of small volume, and for large-scale screening of allergens.

2. The Prior Arts

Allergy (or hypersensitivity) refers to an abnormal sensitivity of the immune system to a substance, which is normally tolerated by human bodies and considered to be harmless. The unusual immune responses may result in some clinical symptoms of allergy. In some cases, such allergic reactions may become life-threatening.

Most of ordinary allergic reactions are categorized as Type I allergy (Immediate Hypersensitivity Reaction). The specificity of the immediate hypersensitivity reaction is attributed to a specific IgE molecule, whose production is provoked by the first contact of human body with a specific allergen. After a second encounter, the allergen will bind to the specific IgE within minutes, activate the mast cells and the basophiles to trigger the release of some mediators, such as histamine and leukotrienes, and consequently result in some allergic symptoms. Possible biological responses to mediators include vasodilation, bronchia tube spasm, or migration of inflammatory cells to inflamed sites, consequently cause allergic symptoms such as sneeze, runny nose, skin rash, itchy eyes, and short breathing. In addition to the allergic mechanisms described above, IgG and IgM antibody are also involved in some allergic reactions via a different mechanism of immune reaction to trigger allergic reactions.

The incidence of allergies is increasing in recent years. In Taiwan, almost one third of the population has allergic characters and suffers symptoms such as food allergy, allergic conjuncitivitis, skin allergy, sinusitis, and asthma. Allergy more or less causes ailment and affects life quality, and are even fatal in some cases. Therefore, identifying the allergens triggering allergic reaction and consequently avoiding the contact of the allergens are the most effective ways to release anaphylaxis. Furthermore, precise detection of the allergen is also useful to hyposensitization treatment to reduce or eliminate allergic symptoms of a patient.

Conventional allergen detection methods commonly employ skin testing or detection of antibody in serum to determine suspicious allergens.

The skin testing involves the injection of a low-dosage allergen to the subcutaneous tissues for observing the allergic reaction. Although the skin testing has the advantages of high accuracy and low cost, it may cause uncomfortable feelings or even an allergic shock. It is also very time consuming for testing many allergens.

As for serum test, immunoassay is routinely employed to determine the concentration of allergy-related antibodies in serum including total IgE, specific IgE, and specific IgG and consequently the hypersensitivity level to a specific allergen is determined. Commonly used immunoassays include enzyme-linked immunosorbent assay (ELISA), radioimmunoassay (RIA), fluorescent immunoassay (FIA), and chemiluminescent assay (CLA). A large volume of samples is required using those immunoassays, and even more for large-scale screening of various allergens. In additions, those assays have the drawbacks of time-consuming and operation-complexity. US Patent Publication No 2003/0109067 provides a method of quantitative detection of allergens in a small-volume sample. However, the allergen labeling process mentioned there is cumbersome. Thus, it is inconvenient and time-consuming to use the method when a variety of allergens need to be determined.

Biochip technology offers advantages of potential applications in diagnosis such as allergy testing, because only a small volume of specimen and reagent is required, and large-scale screening and detection can be achieved within a short time. For examples, WO 02/29415 and US 2003/0073249 disclosed methods using biochip to detect allergens. WO 02/29415 disclosed a detection system using a sequential binding of immobilized allergen, immunoglobulin of interest in sample, and secondary antibody labeled with fluorescencent substance to the solid substrate. The concentration of the captured immunoglobulin is determined by detecting the signal generated by fluorescencent substance labeled on the secondary antibody. US 2003/0073249 disclosed another microarray allergen detection system, using a sequential binding of immobilized allergen, immunoglobulin of interest in sample, secondary antibody labeled with biotin, and streptavidin labeled fluorescencent substance onto the solid substrate to detect the concentration of immunoglobulins. The signal emitted by the fluorescencent substance labeled on streptavidin is used for concentration determination. However, both methods described above are restricted to qualitative but not quantitative determination. That is, these methods can only be used to identify whether a person is allergic to some specific allergens or not, but the hypersensitivity level is still not certainly determined yet.

In the aspect of clinical practice, there is a great variance in kinds of allergens and hypersensitivity levels among allergic individuals. More available information will allow physicians to provide more adequate medication and desensitization treatment. Based on the above mention, there is a need to improve the method for allergen detection. A method with advantages of providing more accurate information, allowing more allergens simultaneously detected in one assay, quantitatively detecting both total IgE and allergen-specific IgE, determining hypersensitivity levels, and requiring minimal sample volume and short detection time will be more beneficial for allergy diagnosis.

### SUMMARY OF THE INVENTION

A primary objective of the present invention is to provide a microarray chip, which can be used to quantitatively detect both concentrations of total IgE and allergen-specific immunoglobulins (such as specific IgE, specific IgG, and specific IgM) in patient's serums. The microarray chip requires minimal volume of serum to perform the detection of a lot of allergens (with only 10-25 μl serum sample for more than 150 allergens).

Another objective of the present invention is to provide a method for preparation of a microarray chip that can be applied to quantitative detection of both concentrations of total IgE and allergen-specific immunoglobulins in patient's serums.

A further objective of the present invention is to provide a method for detection of both total IgE and allergen-specific immunoglobulins using a microarray chip, which needs minimal sample volume for the detection of a large variety of allergens. Moreover, the method is very time-effective, featuring detections of 96-150 allergens within 1.5-4 hours, and allows quantitative detection of both total IgE and allergen-specific immunoglobulins. Therefore, the information obtained is useful to determine the hypersensitivity levels for an allergic individual.

The term “secondary antibody” used herein may be an antibody that can specifically interact with the immunoglobulin of interest in samples.

The microarray chip provided in the present invention comprises: a solid substrate to be provided as a supporting substance; a reactive layer fabricated on the solid substrate, wherein the layer comprises at least one reactive amino group to a protein; and at least one substance capable of binding with the immunoglobulins of interest, which is immobilized on the solid substrate via the fabricated reactive layer, and wherein the microarray chip performs a qualitative or quantitative detection of allergy-related immunoglobulins in a sample.

The substance binding to immunoglobulins of interest may be any substance comprising a moiety binding to an immunoglobulin of interest.

For detection of total IgE, the substance preferably binds specifically to the immunoglobulin of interest. For example, if the total concentration of IgE in a sample is to be determinated, the substance is preferably an anti-IgE antibody.

In case that detection of allergen-specific immunoglobulins, the substance may be replaced by an allergen of interest. There is no limitation on the allergens used in the present invention. The allergens may be common allergens used in the current allergen tests, or a specific allergen for a sample of interest.

The solid substrate may be glass, plastics, or metal. It is preferably glass.

The microarray chip may comprise a plurality of allergen spots, the density on which is at least 300-484 allergen spots/cm². At least 150 allergens can be detected in the same time if each allergen is detected in duplicate or triplicate on the microarray chip.

A method for preparing the microarray chip comprises the steps of:
(a) preparing a solid substrate;
(b) fabricating reactive layer on the solid substrate, wherein the layer comprises at least one reactive amino group to the substance;
(c) preparing a solution comprising a substance capable of binding to an immunoglobulin of interest at a predetermined concentration;
(d) spotting the solution on the reactive layer at a predetermined matrix density;
(e) allowing the substance to interact with the reactive layer and subsequently being immobilized on the solid substrate; and
(f) inactivating the residual functional group of the reactive layer.

To accurately perform spotting, an instrument for printing microarrays may be applied to spot the solution(s) containing the substances capable of binding to an immunoglobulin of interest on the microarray chip. The optimal conditions, such as temperature and humidity for carrying out the spotting, are dependent on the spotting solutions. Preferably, the relative humidity is in the range of 40 to 90%.

The immobilization of the substance on the reactive solid substrate is taken place under a high humidity condition for a period of time. Subsequently, a conventional blocking buffer, such as a PBST buffer (phosphate buffer solution with Tween 20) containing bovine serum albumin, is added and allowed to inactivate the surface at room temperature to 42° C. for 15 min to one hour.

Also, the substance may be replaced by specific allergens to prepare a microarray chip for detection of allergen-specific immunoglobulins.

Allergy-related immunoglobulin detection using the microarray chip of the present invention comprises the steps of:
(i) providing a microarray chip on which at least one substance capable of binding to an immunoglobulin of interest is immobilized;
(ii) contacting a sample with the microarray chip and allowing the substance to bind to the immunoglobulin in the sample;
(iii) removing the immunoglobulin which is not bound to the substance after a predetermined period of time;
(iv) allowing the immunoglobulin bound on the chip to bind to a secondary antibody, on which a linking portion capable of binding to a signal generation unit is linked;
(v) removing the secondary antibody that is not bound to the immunoglobulin after a predetermined period of time;
(vi) allowing the linking portion on the secondary antibody to bind to a signal generation unit;
(vii) removing the signal generation unit that is not bound to the linking portion after a predetermined period of time;
(viii) allowing the signal generation unit to generate a signal; and
(ix) measuring the signal to determine the concentration of allergy-related immunoglobulins in the sample.

The secondary antibody for the detection may be a monoclonal antibody or polyclonal antibody, and preferably a monoclonal antibody. The binding of monoclonal antibody is more specific and thus avoidance of interference derived from non-specific binding of polyclonal antibody can be achieved.

Furthermore, the linking portion capable of binding to a signal generation unit on the secondary antibody herein may be biotin. The signal generation unit comprises a moiety to bind to the linking portion on secondary antibody (for example, the moiety may be streptavidin if the linking portion is biotin) and a portion to generate signals. The preferred signal generation portion is an enzyme, for examples, horesradish peroxidace (HRP), hydroperoxidase, alkaline phosphorase, or β-galactosidase, etc. Substrate labeled with fluorescent substances, for examples, Alexa Flour 647 tyramide, Alexa Flour 546 tyramide, Alexa Flour 532 tyramide, Cy3 tyramide or Cy5 tyramide, can be used in the enzyme reaction to generate signals. The enzymatic reaction amplifies the binding signals derived from the immunoglobulin of interest and the secondary antibody makes quantification of the immunoglobulin possible. Alexa Flour is a preferred fluorescent substance to label substrate because of its high quantum yield.

Referring to FIG. 1, the above-mentioned detection method is clearly presented. As shown in FIG. 1(a), a microarray chip (1) with immobilized substances capable of binding to immunoglobulin of interest (2) on its surface is provided; in FIG. 1(b), the sample solution is allowed to contact with the surface of the microarray chip, the immunoglobulin of interest (3) in the sample solution consequently binds to the substances immobilized on the microarray chip; and in FIG. 1(c), a secondary antibody (41) is allowed to bind to the immunoglobulin of interest (3), and the secondary antibody (41) links a portion (42) capable of binding to a signal generation unit; as shown in FIG. 1(d), the signal generation unit is allowed to bind to the linking portion (42) on secondary antibody (41) by the moiety (51), the signal generation unit is labeled with an enzyme (52); as shown in FIG. 1(e), the enzyme reaction is carried out by adding substrate (6) labeled with fluorescent substance, and the fluorescent signals are generated; the signal is measured and the concentration of the immunoglobulin of interest (3) is determined.

In order to detect the concentration of allergen-specific immunoglobulins, the above-mentioned substance may be replaced by the allergen of interest.

As described above, to quantitatively measure the allergy-related immunoglobulin (antibody), the present invention uses a highly specific monoclonal secondary antibody and a multiple signal amplification system, thus realizing the quantitative measurement.

The present invention will be further elaborated in detail by the following examples and drawings. Those who are skilled in the art may modify the present invention upon review of this specification, but the new discovery is still within the scope of the present invention. These following examples should not, however, be considered to limit the scope of the invention, which is defined by the appended claims.

### BRIEF DESCRIPTION OF THE DRAWINGS

The related drawings in connection with the detailed description of the present invention to be made later are described briefly as follows, in which:

FIG. 1 shows schematic diagram of allergy-related immunoglobulin detection using a microarray chip of the present invention.

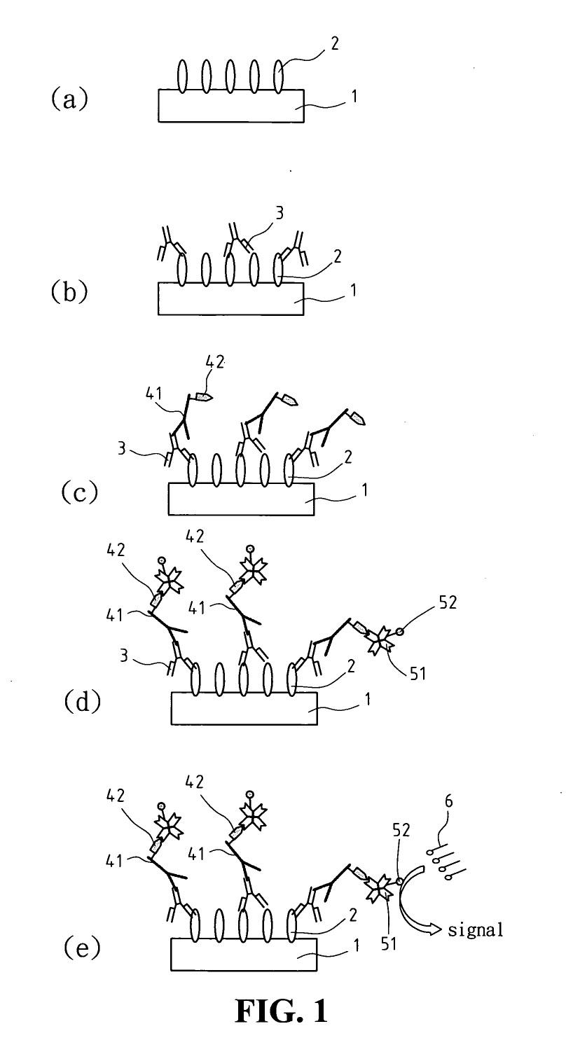

FIG. 2 shows the relationship between the resulting microarray intensities to spotted anti-IgE and the concentrations of pure total IgE; W21 and W23 are used as negative and positive controls, respectively. The microarray intensities were obtained by using standard total IgE of known concentrations as assayed samples. The obtained regression equation can be subsequently used to determine the total IgE concentration of an unknown sample by interpolating.

FIG. 3 shows the relationship between the resulting microarray intensities to spotted D. farinae extracts and the concentrations of serum D. farinae-specific IgE determined using UniCAP. The obtained regression equation can be subsequently used to determine the D. farinae-specific IgE concentration of an unknown sample by interpolating.

FIG. 4. shows the relationship between the resulting microarray intensities to spotted D. pteronyssinus extracts and the concentrations of serum D. pteronyssinus-specific IgE determined using UniCAP. The obtained regression equation can be subsequently used to determine the D. pteronyssinus-specific IgE concentration of an unknown sample by interpolating.

FIG. 5 shows the relationship between the resulting microarray intensities to spotted B. tropicals extracts and the concentrations of serum B. tropicals-specific IgE determined using UniCAP. The obtained regression equation can be subsequently used to determine the B. tropicals-specific IgE concentration of an unknown sample by interpolating.

### DETAILED DESCRIPTION OF THE PREFERRED EMBODIMENT

### Example 1

### Preparation of Microarray Chip for Allergen-specific Immunoglobulin Detection

An allergen solution is prepared by using either commercially available products or the extract isolated from allergen raw materials. The allergen solution is prepared at concentrations of 0.01-10 mg/ml depending on allergens. Glycerol might be added in a final concentration of 5-50% to facilitate the subsequent immobilization process and preservation.

A glass substrate is provided as a solid substrate and coated with a layer of amine-terminated silane to act as a reactive layer to allergens.

By means of a microarray instrument, allergens are spotted onto the surface of the amine-terminated solid substrate. The allergen solution is spotted as a small dot of about 150 μm in diameter, with each dot about 300 μm apart. Depending on the number of allergens tested, a spot density of at least 300-484 dots/cm² can be achieved. Spotting is carried out at 40-90% relative humidity and room temperature. After spotting, the microarray chip is incubated at a high humidity condition for a period of time in order to immobilize the allergen(s). Then PBST buffer solution containing 3% of BSA is added and allowed to block residual unreacted amino functional group(s) in the reactive layer for 15 min to 1 h. A microarray chip for allergen-specific immunoglobulins detection is prepared and ready for the detection.

### Example 2

### Allergen-specific IgE Detection Using Microarray Chip of the Present Invention

A microarray chip for allergen-specific IgE detection is prepared according to the example 1. A 2-10 folds diluted serum sample is put in contact with the microarray chip and incubated at room temperature to 42° C. to allow the binding of serum IgE with the spotted allergen. After 15 min to one hour, the microarray chip is washed with a buffer solution (for examples, PBS, PBST, or TBST (tris phosphate buffer)) to remove unreacted reagents. Then, a solution of anti-IgE monoclonal antibody conjugated with biotin is added and incubated for another 15 min to one hour. The microarray chip is subsequently washed again with a buffer solution to remove unreacted anti-IgE monoclonal antibody.

Next, a HRP-streptavidin conjugate is added and incubated to bind with the captured biotin-conjugated antibody. After 15 min to one hour incubation, unreacted HRP-streptavidin conjugate is washed off using the buffer solution. Subsequently, Alexa 647 tyramide is added as a substrate to perform the colorimetric reaction. After the reaction, excess substrate is removed by washing and the microarray chip is dried at room temperate. A laser scanner (GenePix 4000B) is then used to measure the fluorescent signal of each spot and the whole detection can be completed within 1.5-5 hours. The concentration of IgE antibody in each serum sample can be finally determined according to the calibration curve of the allergen-specific IgE, which is obtained following the procedures described in the example below.

### Example 3

### Establishment of Calibration Curve and Detection of Total IgE Concentration in Serum Samples

A microarray chip for total IgE concentration detection is prepared according to the example 1 except that a polyclonal anti-IgE antibody is spotted on the reactive layer of the microarray chip. Next, the microarray chip is used to react with various concentrations of hIgE, following the procedure described in Example 2 with the exception of replacement of serum samples with the standard hIgE solutions. Standard hIgE solutions at 5, 25, 100, 500, and 2500 IU/ml concentrations are used to obtain the calibration curve.

In addition, two serum samples, designated as W21 and W23, are also assayed as a negative and a positive serum sample, respectively. The total IgE concentrations of W21 and W23 were predetermined as 12.8 and 1522 IU/ml, respectively, using a commercial product (UniCAP-100, Sweden Pharmacia Diagnostics Inc.).

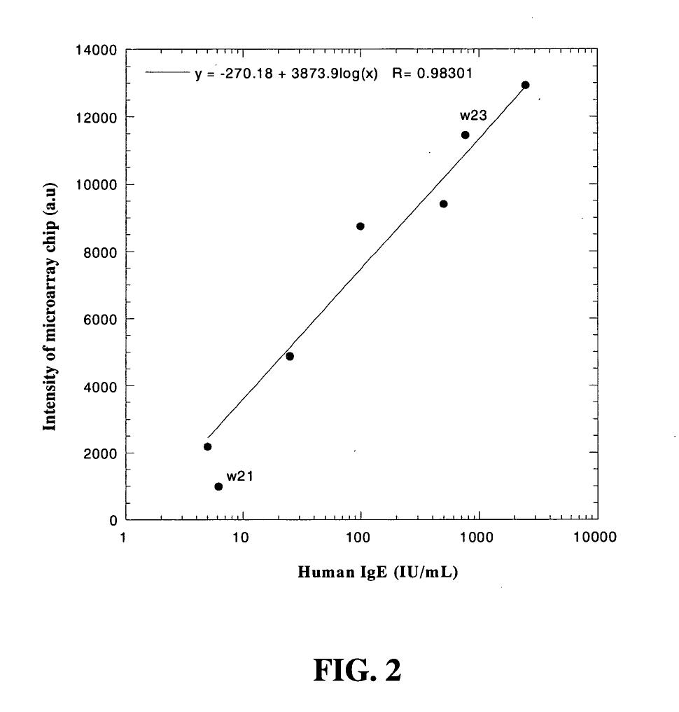

The result shown in FIG. 2 indicates that the detected microarray signals are well correlated with the standard hIgE concentrations (the R value (regression coefficient) is 0.98301) and that the points of W21 and W23 are located close to the regression curve. Therefore, the curve can be used as a calibration or standard curve to determine the concentration of total IgE in an unknown sample.

### Example 4

### Quantitative Detection of D. farinae-specific IgE Using a Microarray Chip

The results of quantitative analysis described herein is compared to the allergen-specific IgE concentrations of serum samples, which are determined by using a commercial instrument (UniCAP-100, Sweden Pharmacia Diagnostics Inc.).

A microarray chip for the detection is prepared according to the example 1 except that the allergen spotted on the solid substrate is the crude extract or recombinant allergens of D. farinae. According to the method described in Example 2, the microarray chip is used to detect 32 serum samples. Meanwhile, the same serum samples are also analyzed by using UniCAP-100. A linear regression analysis of the results obtained from both assays is carried out.

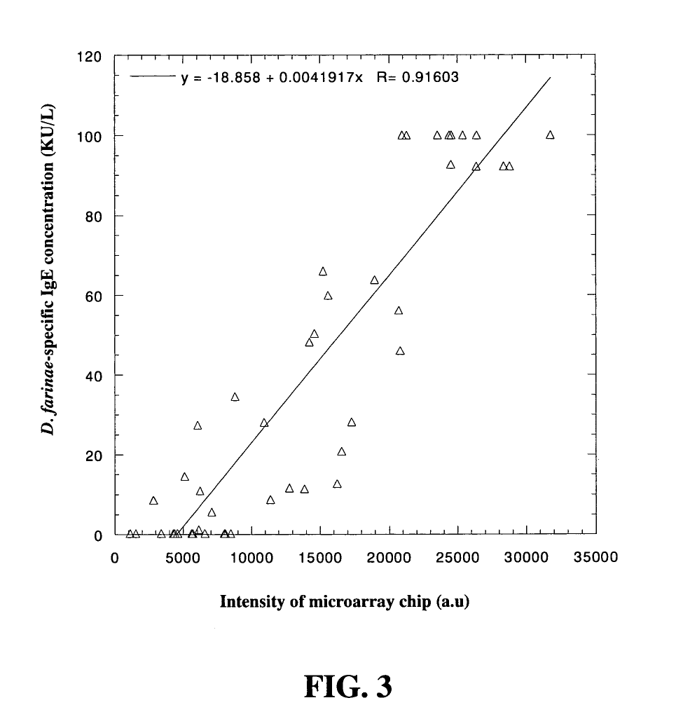

Referring to FIG. 3, the analysis results of 32 serum specimens by the present microarray assay and by using UniCAP-100 yield a curve with a high R value 0.91603, indicating that there is high correlation between these two analyses and that their results are well comparable with each other. It confirms that the method of the present invention can be used for quantitative detection of D. farinae-specific IgE in a serum sample. Then the hypersensitivity level of the serum sample can be determined according to the estimated allergen-specific concentration.

### Example 5

### Quantitative Detection for D. pteronyssinus-specific IgE Using a Microarray Chip

A microarray chip for the allergen detection is prepared according to example 1 except that the allergen spotted on the solid substrate is the crude extract or recombinant allergens of D. pteronyssinus. The microarray chip is used to detect 32 serum samples, following the procedures described in Example 2. In addition, the same serum samples are also analyzed by using UniCAP-100. A linear regression analysis of the results obtained from both assays is carried out.

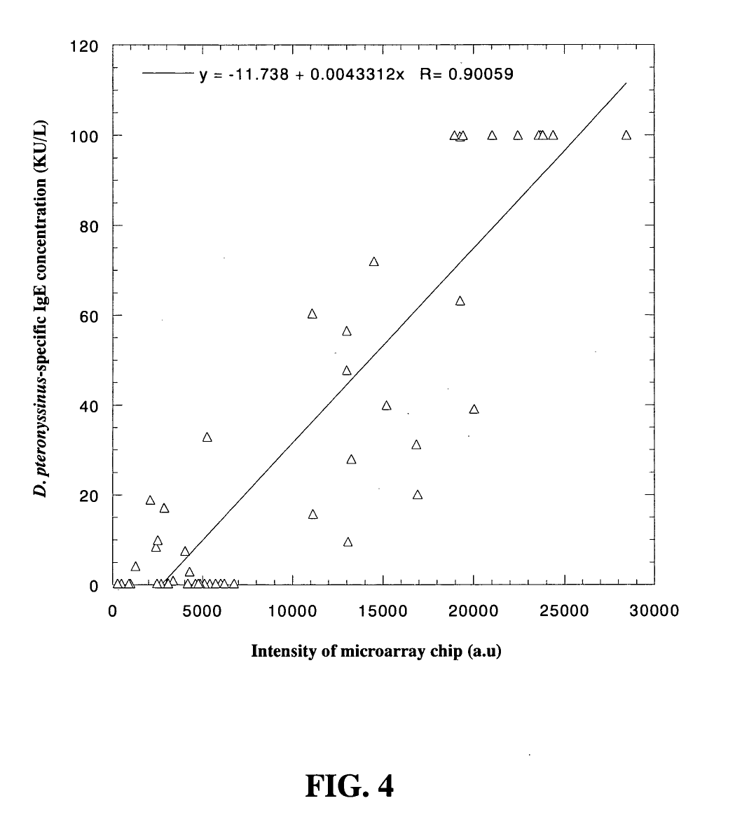

Referring to FIG. 4, the analysis results of 32 serum specimens by the present microarray assay and by using UniCAP-100 yield a curve with a high R value 0.90059, indicating that there is high correlation between these two analyses and that their results are well comparable with each other. It confirms that the method of the present invention can be used for quantitative detection of D. pteronyssinus-specific IgE in a serum sample. Then the hypersensitivity level of the serum sample can be determined according to the estimated, allergen-specific concentration.

### Example 6

### Quantitative Detection for B. tropicals-Specific IgE Using a Microarray Chip

A microarray chip for the allergen detection is prepared according to example 1 except that the allergen spotted on the solid substrate is the crude extract or recombinant allergens of B. tropicals. The microarray chip is used to detect 32 serum samples, following the procedures described in Example 2. In addition, the same serum samples are also analyzed by using UniCAP-100. A linear regression analysis of the results obtained from both assays is carried out.

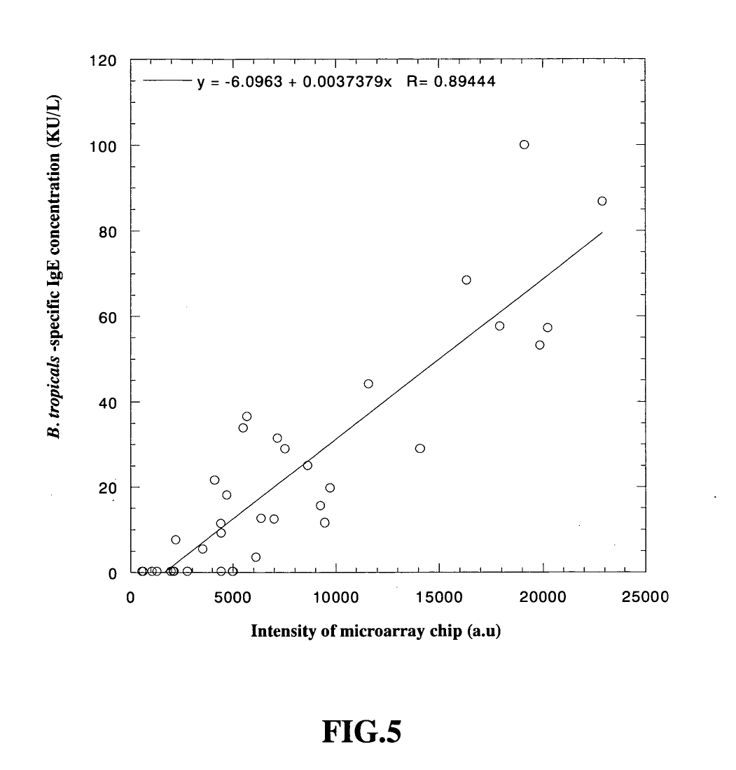

Referring to FIG. 5, the analysis results of 32 serum specimens by the present microarray assay and by using UniCAP-100 yield a curve with a high R value 0.89444, indicating that there is high correlation between these two analyses and that their results are well comparable with each other. It confirms that the method of the present invention can be used for quantitative detection of B. tropicals-specific IgE in a serum samples. Then the hypersensitivity level of the serum sample can be determined according to the estimated allergen-specific concentration.

In addition to the abovementioned examples, we also compare features of the microarray chip of the present invention to commercial products, such as UniCAP and MAST (manufactured by Hitachi), in respect to amount of serum volume required, detection cost, capability of total IgE concentration determination, capability of quantitative analysis, and time consumed for detection. The results are listed in Table 1.

Table 1 shows that the microarray chip provided in the present invention carries out more tests with less cost and less time by a smaller sample volume in comparison with the commercial products. In addition, the present invention provides quantitative detection and the detection result is well comparable to the results using the commercial products. Therefor, it has a great potential for practical application.

According to the description above, the present invention provides a microarray chip easy to prepare and a method for total hIgE and allergen-specific immunoglobulins detection, which allows large-scale screening of eliciting allergens within a short time using a minimal sample volume.

TABLE 1
Comparison of microarray chip of the present invention
with commercial product UniCAP and MAST

| Item | Present invention | UniCAP | MAST |
|---|---|---|---|
| Required serum | 25 μl/96-150 tests | 150 μl/per test | 1300 μl/36 tests |
| volume | | | |
| Detection cost | About | About | About |
| | ≧NT$110 (US$3.3)/96 tests | NT$180 (US$5.4)/ | NT$1500 (US$44.9)/36 tests; |
| | | test; total cost for 96 | total cost for 96 tests is about |
| | | tests is about | NT$ 4500 (US$134.7) |
| | | NT$17280 (US$ 517) | |
| Determination of | Yes | Yes | No |
| total IgE | | | |
| concentration | | | |
| Quantitative | Yes (quantitative and | Yes (quantitative and | Determine hypersensitivity |
| analysis | determine | determine | level. |
| | hypersensitivity level.) | hypersensitivity level.) | |
| Time | 1.5-4 hrs/96-150 tests | 8 hrs/88 tests | >16 hrs/36 tests |
| consumed | | | |

What is claimed:

1. A microarray chip, which is for allergy detection, wherein the microarray chip comprising:
a solid substrate providing as a supporting substance;
a reactive layer fabricated on the solid substrate, which layer comprises at least one reactive group to a protein; and
at least one substance capable of binding with the immunoglobulins of interest, which is immobilized on the solid substrate via the fabricated reactive layer;
wherein the microarray chip performs a quantitative detection of total IgE or an allergen-specific immunoglobulins in a sample.

2. The microarray chip as claimed in claim 1, wherein the substance is an anti-immunoglobulin antibody to IgE or allergens to the allergen-specific immunoglobulins.

3. The microarray chip as claimed in claim 1, wherein the solid substrate is glass, plastics, or metal.

4. The microarray chip as claimed in claim 1, wherein the allergen-specific immunoglobulins are specific IgE, specific IgG, or specific IgM

5. A method for preparing a microarray chip for allergy detection, comprising the steps of:
(a) preparing a solid substrate;
(b) fabricating a reactive layer on the solid substrate, which layer comprises at least one reactive group to the substance;
(c) preparing a solution comprising at least one substance or one allergen capable of binding to an immunoglobulin of interest at a predetermined concentration;
(d) spotting the solution on the reactive layer at a predetermined matrix density;
(e) allowing the substance to interact with reactive layer and subsequently be immobilized on the solid substrate; and
(f) inactivating the residual functional group of the reactive layer.
wherein the microarray chip performs a quantitative detection of total IgE or an allergen-specific immunoglobulins in a sample.

6. The method as claimed in claim 5, wherein the matrix density is at least 300-484 dots/cm².

7. The method as claimed in claim 5, wherein the spotting step, step (d), is performed with a spot-printing instrument.

8. The method as claimed in claim 5, wherein step (d) is performed under a relative humidity of 40%-90%.

9. The method as claimed in claim 5, wherein a blocking buffer is applied in step (f) to inactivate the residual functional group of the reactive layer.

10. A method for quantitative measurement of total IgE or allergen-specific immunoglobulins with a microarray chip, comprising the steps of:
(i) providing a microarray chip on which at least one substance or one allergen capable of binding to an immunoglobulin of interest is immobilized;
(ii) contacting a sample with the microarray chip and allowing the substance or the allergen to bind to the immunoglobulin in the sample;
(iii) removing the immunoglobulin which is not bound to the substance or the allergen after a predetermined period of time;
(iv) allowing the immunoglobulin bound on the chip to bind to a secondary antibody, on which a linking portion capable of binding to a signal generation unit is linked.
(v) removing the secondary antibody that is not bound to the immunoglobulin after a predetermined period of time;
(vi) allowing the linking portion on the secondary antibody to bind to a signal generation unit;
(vii) removing the signal generation unit that is not bound to the linking portion after a predetermined period of time;
(viii) allowing the signal generation unit to generate a signal; and
(ix) measuring the signal to determine the concentration of total IgE or allergy-related immunoglobulins in the sample.

11. The method as claimed in 10, wherein the secondary antibody is a polyclonal or monoclonal antibody.

12. The method as claimed in 10, wherein the linking portion on the secondary antibody is biotin.

13. The method as claimed in claim 12, wherein the binding in step (vi) is binding of streptavidin to biotin.

14. The method as claimed in claim 10, wherein the signal generation unit comprises an enzyme.

15. The method as claimed in claim 14, wherein the enzyme is selected from a group consisting of hydroperoxidase, horseradish peroxidase, alkaline phosphorase, and β-galactosidase.

16. The method as claimed in claim 10, wherein the signal generated in step (viii) is measured after an enzyme reaction.

17. The method as claimed in claim 16, wherein a substrate for the enzyme reaction is fluorescein-labeled.

18. The method as claimed in claim 17, wherein the fluorescien is selected from a group consisting of Alexa fluorescent dye, Cy3 and Cy5.

19. The method as claimed in claim 18, wherein the Alexa fluorescent dye is selected from a group consisting of Alexa Fluor 647, Alexa Fluor 546 and Alexa Fluor 532.
